SEQUENCE LISTING (26 sequences)

SEQ ID NO: 1 - DNA, 12 bp, Artificial
  Synthetic
cccccccccctt

SEQ ID NO: 2 - DNA, 37 bp, Artificial
  Synthetic
ggtaatacgactcactatagcgagacccccccccctt

SEQ ID NO: 3 - DNA, 30 bp, Artificial
  Synthetic
aaaaaaaaaaaaaaaaaaaagngngngngn

SEQ ID NO: 4 - DNA, 27 bp, Artificial
  Synthetic
ccccctttttttttttttttttttttt

SEQ ID NO: 5 - DNA, 19 bp, Artificial
  Synthetic
aaaaaaaaagggnggnggg

SEQ ID NO: 6 - DNA, 11 bp, Artificial
  Synthetic
ccccccccctt

SEQ ID NO: 7 - DNA, 18 bp, Artificial
  Synthetic
cccccccccttttttttt

SEQ ID NO: 8 - DNA, 18 bp, Artificial
  Synthetic
aaaaaaaaagggnggngg

SEQ ID NO: 9 - DNA, 22 bp, Artificial
  Synthetic
aaaaaaaaaaaaaaaaaaaaaa

SEQ ID NO: 10 - DNA, 28 bp, Artificial
  Synthetic
cccccctttttttttttttttttttttt

SEQ ID NO: 11 - DNA, 24 bp, Artificial
  Synthetic
aaaaaaaaaaaaaaaaaaaaaaaa

SEQ ID NO: 12 - DNA, 60 bp, Artificial
  Synthetic
ccccacagagctaactgggttacagtgttttatccgaaagtttccaattccactgtcttg

SEQ ID NO: 13 - DNA, 60 bp, Artificial
  Synthetic
tgttttcatgttgaaaatacttttgcatttttcctttgagtgccaatttcttactagtac

SEQ ID NO: 14 - DNA, 60 bp, Artificial
  Synthetic
tatttcttaatgtaacatgtttacctggaatgtattttaactatttttgtatagtgtaaa

SEQ ID NO: 15 - DNA, 60 bp, Artificial
  Synthetic
ctgaaacatgcacattttgtacattgtgctttcttttgtgggacatatgcagtgtgatcc

SEQ ID NO: 16 - DNA, 60 bp, Artificial
  Synthetic
agttgttttccatcatttggttgcgctgacctaggaatgttggtcatatcaaacattaaa

SEQ ID NO: 17 - DNA, 60 bp, Artificial
  Synthetic
aatgaccactcttttaattgaaattaacttttaaatgtttataggagtatgtgctgtgaa

SEQ ID NO: 18 - DNA, 60 bp, Artificial
  Synthetic
gtgatctaaaatttgtaatatttttgtcatgaactgtactactcctaattattgtaatgt

SEQ ID NO: 19 - DNA, 36 bp, Artificial
  Synthetic
aaaaaaaaaaaaaaacagtgacattgataaaaataa

SEQ ID NO: 20 - DNA, 24 bp, Artificial
  Synthetic
ccacagagctaactgggttacagt

SEQ ID NO: 21 - DNA, 23 bp, Artificial
  Synthetic
tgtaacatgtttacctggaatgt

SEQ ID NO: 22 - DNA, 26 bp, Artificial
  Synthetic
tgtatagtgtaaactgaaacatgcac

SEQ ID NO: 23 - DNA, 24 bp, Artificial
  Synthetic
cattgtgctttcttttgtgggaca

SEQ ID NO: 24 - DNA, 24 bp, Artificial
  Synthetic
tggttgcgctgacctaggaatgtt

SEQ ID NO: 25 - DNA, 20 bp, Artificial
  Synthetic
cgctgacctaggaatgttgg

SEQ ID NO: 26 - DNA, 23 bp, Artificial
  Synthetic
gtcactgtaactatttttattac

(19) United States
(12) Patent Application Publication
(10) Pub. No.: US 2011/0059453 A1
(43) Pub. Date: Mar. 10, 2011

(54) Poly(A) Tail Length Measurement by PCR

(75) Inventors: Sangpen Chamnongpol, Twinsburg, OH (US); Christopher J. Kubu, Twinsburg, OH (US)

(73) Assignee: AFFYMETRIX, INC., Santa Clara, CA (US)

(21) Appl. No.: 12/861,783

(22) Filed: Aug. 23, 2010

Related U.S. Application Data
(60) Provisional application No. 61/236,128, filed on Aug. 23, 2009.

(51) Int. Cl. C12Q 1/68; C07H 21/00

(52) U.S. Cl. 435/6; 536/24.33

(57) ABSTRACT

Methods and kits for measuring the length of the poly(A) tail of selected target mRNA are disclosed herein. In preferred aspects the mRNA population is modified by addition of a tail comprising guanosine and inosine (G/I tailing). The added tail is used as a priming site for reverse transcription. The resulting cDNA is then amplified using one or more target specific primers and a universal primer that recognizes the tailed region. The products are separated according to size and the size is used to estimate the polyA tail length.

### RELATED APPLICATION

This application claims the benefit of U.S. provisional application 61/236,128, filed on Aug. 23, 2009, which is herein incorporated by reference in its entirety for all purposes.

### FIELD OF THE INVENTION

The invention is related to methods for measuring the polyA tail length for selected mRNAs.

### BACKGROUND OF THE INVENTION

The poly-adenylated(A) tail on most eukaroyotic mRNAs plays a number of important roles in mRNA metabolism including enhancing translation, mRNA stability and transport from the nucleus. Recent studies in a variety of model organisms have revealed a pivotal role for regulated deadenylation as being rate limiting for mRNA degradation and importantly deadenylation is now recognized as a mechanism of miRNA mediated gene regulation. This heightened interest in poly(A) metabolism encouraged us to develop convenient and powerful methods to measure poly(A) tail length. Current available techniques include ligation-mediated PCR and Northern blotting, both of which are limited to analysis of abundant mRNAs.

In eukaryotic cells, mRNA is predominately degraded by two alternative pathways that are both initiated by shortening of the 3′ polyadenosine tail (deadenylation). Following deadenylation, either the 5′ 7mGpppN cap is removed (decapping) and the message is digested exonucleolytically 5′ to 3′ or alternatively the transcript is destroyed 3′ to 5′ by the cytoplasmic exosomel. The two mechanisms of mRNA decay together determine basal mRNA levels thereby significantly contributing to overall gene expression.

### SUMMARY OF THE INVENTION

In preferred aspects the methods utilize the capacity of yeast poly(A) polymerase (yeast PAP) to add a limited number (˜25) of guanosine residues to an existing poly(A) tail. This polymerase also can add inosine. This is an advantage for the disclosed methods because inosine is less likely than guanosine to form self-pairing structures. In preferred aspects, total RNA is extended with a mixture of guanosine and inosine to generate a G/I tail. This tail then provides a priming site for reverse transcription. When coupled with a gene specific internal primer, the methods allow for quick and accurate determination of the length distribution of poly(A) tails in target mRNAs. The disclosed methods should be of broad utility in the field of analysis of poly(A) tail metabolism.

### BRIEF DESCRIPTION OF THE DRAWINGS

FIG. 1 is a schematic of a method of poly(A) tail length measurement by PCR.

FIG. 2A is a schematic of the assay showing exemplary sequences.

FIG. 2B shows how the results vary in different samples.

FIG. 3 is a schematic the sensitivity of the assay using dilutions of HeLa RNA.

FIGS. 4A and B show the results of the stability testing after 1 month storage at −20° C. for poly(A) tail detection (4A) and gene specific detection (4B).

FIG. 5 shows stability testing after 7 weeks storage at −20° C. and 17 days storage at 4° C. Actin, k-ras, and nucleolin were tested using 1 μg HeLa total RNA.

FIG. 6 shows the results of a test of a HOTSTART-IT PCR master mix in the assay.

FIG. 7 shows the results of testing different PCR components and amplification conditions for this assay. One μg HeLa total RNA was used. Different amounts of Mg²⁺, and HotStart-IT taq were tested and a 2 step PCR was compared to a 3 step PCR.

FIG. 8 shows results on a sample depleted of poly(A) by RNaseH treatment.

FIG. 9 shows assay results for different RNA samples and under different conditions.

FIG. 10 shows assay results for different RNA samples and under different conditions.

FIG. 11 is another schematic of the methods.

FIG. 12 shows schematically how the length of the poly(A) tail is calculated.

FIG. 13 shows exemplary results.

FIG. 14 shows exemplary results.

FIG. 15 shows results using different specific forward primer designs for poly(A) tail-length determination. FIG. 15A shows the gel results. FIG. 15B shows the primer binding sites and FIG. 15C shows the primer sequences.

### DETAILED DESCRIPTION OF THE INVENTION

Reference will now be made in detail to exemplary embodiments of the invention. While the invention will be described in conjunction with the exemplary embodiments, it will be understood that they are not intended to limit the invention to these embodiments. On the contrary, the invention is intended to cover alternatives, modifications and equivalents, which may be included within the spirit and scope of the invention.

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being, but may also be other organisms including, but not limited to, mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “hybridization” as used herein refers to the process in which two single-stranded polynucleotides bind noncovalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2^{nd} Ed. Cold Spring Harbor Press (1989) which is hereby incorporated by reference in its entirety for all purposes above.

The term “label” as used herein refers to a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

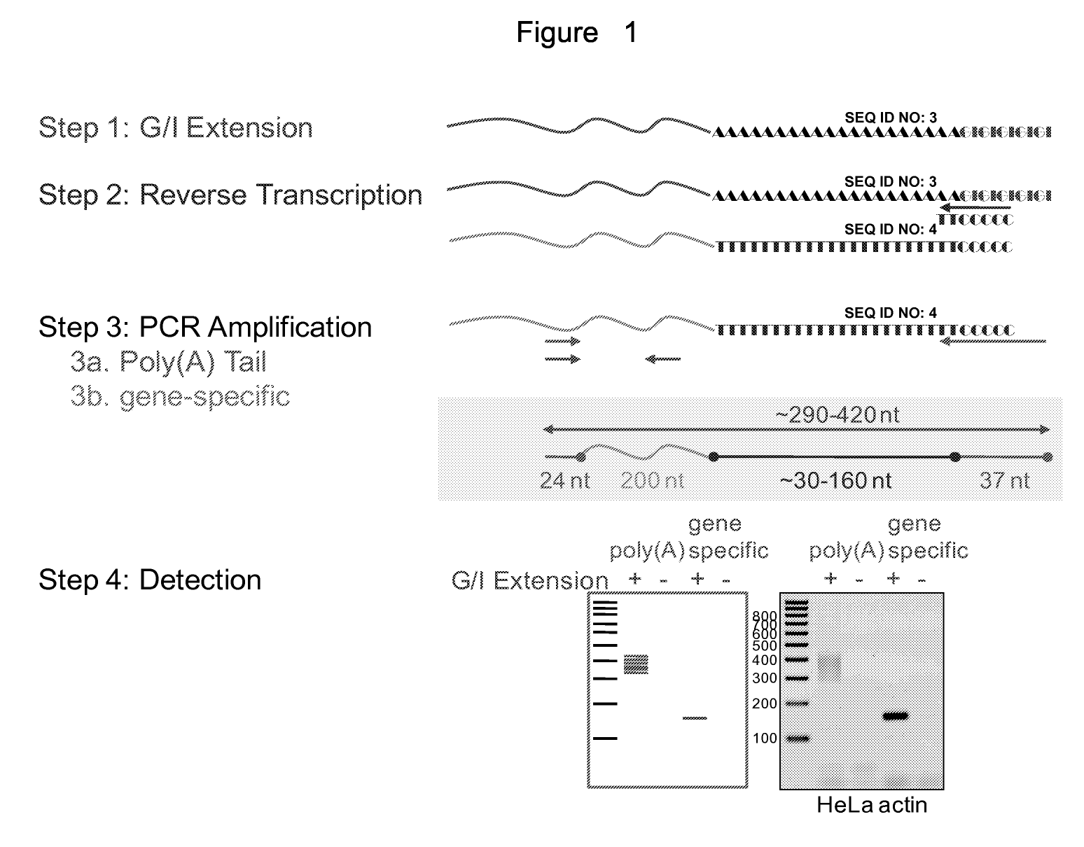

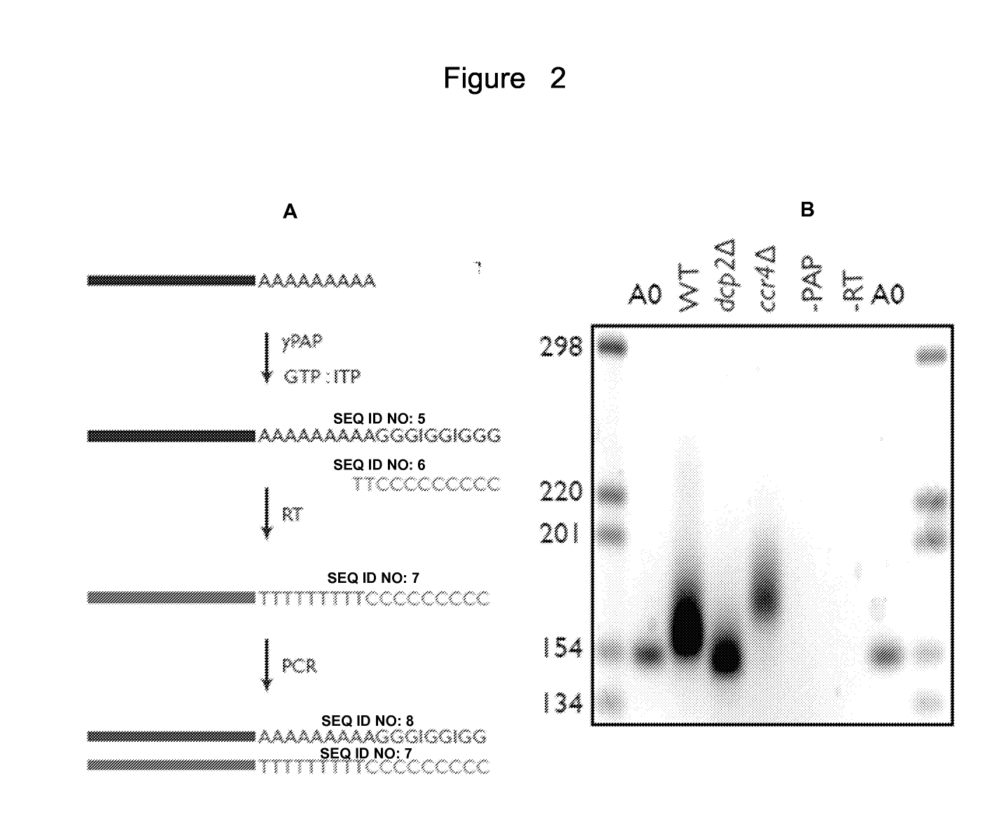

A preferred aspect of the method is shown schematically in FIG. 1. In general there are three steps. The first step includes extending the poly(A) tail using PAP in the presence of G and I. The second step includes performing reverse transcription using a poly C primer. The third step includes performing PCR amplification using a target specific primer. For example, step 1 may be Poly(A) tail extension with G/I—1 hr incubation. Step 2 may be Reverse Transcription using a 1 hr 10 min incubation. Step 3 may be PCR amplification including a 30 min to 1 hr incubation In a preferred aspect the primer used for the reverse transcription (RT) reaction, is selected so that it does not work in the PCR amplification (Table 1). Preferably, a universal PCR reverse primer is used for this assay. This allows for generation of only the expected PCR products using optimal assay conditions for both RT and PCR reactions. Although using the same primer for RT and PCR reactions would result in poly(A) tail length detection, the carry-over RT primer could generate non-specific PCR amplification in one of the required controls for this assay. In a preferred aspect the RT primer is a C10T2 primer: 5′-CC CCC CCC CCT T-3′ (SEQ ID NO: 1) and the universal PCR Reverse Primer—anchor is 25C10T2: 5′-GGT AAT ACG ACT CAC TAT AGC GAG ACC CCC CCC CCT T-3′ (SEQ ID NO: 2)

The following references are incorporated herein by reference in their entireties. They disclose, for example, background information relating to polyadenylation and analysis of poly(A) tail length and function. He, F. & Jacobson, A., Mol Cell Biol 21 (5), 1515-1530 (2001) which shows that Upf1p, Nmd2p, and Upf3p regulate the decapping and exonucleolytic degradation of both nonsense-containing mRNAs and wild-type mRNAs. Hsu, C. L. & Stevens, A., Mol Cell Biol 13 (8), 4826-4835 (1993), showing that yeast cells lacking 5′ to 3′ exoribonuclease 1 contain mRNA species that are poly(A) deficient and partially lack the 5′ cap structure. Muhlrad, D., Decker, C. J., & Parker, R., Mol Cell Biol 15 (4), 2145-2156 (1995), showing turnover mechanisms of the stable yeast PGK1 mRNA. Couttet, et al. Proc Natl Acad Sci USA 94 (11), 5628-5633 (1997), teaching messenger RNA deadenylylation precedes decapping in mammalian cells. Amrani, et al. Nature 453 (7199), 1276-1280 (2008), teaching translation factors promote the formation of two states of the closed-loop mRNP. Jacobson, A. & Peltz, S. W., Annu Rev Biochem 65, 693-739 (1996), teaching interrelationships of the pathways of mRNA decay and translation in eukaryotic cells. Moore, M. J. & Query, C. C., Methods Enzymol 317, 109-123 (2000), showing joining of RNAs by splinted ligation. Mangus, D. A. & Jacobson, A., Methods 17 (1), 28-37 (1999), showing linking mRNA turnover and translation: assessing the polyribosomal association of mRNA decay factors and degradative intermediates.

The poly-adenylated tail (poly(A) tail) on nearly all eukaroyotic mRNAs plays a number of important roles in mRNA metabolism including enhancing translation, mRNA stability and transport from the nucleus. See, Parker R., and Song H. (2004) Nat Struct Mol Biol. 11, 121-127 and Andersen K. R., Jensen T. H., and Brodersen D. E. (2008) Biochim Biophys Acta. 1779, 532-537. Studies in several model organisms have shown regulated deadenylation is rate limiting for mRNA degradation. Importantly, deadenylation is now recognized as a mechanism of miRNA mediated gene regulation. See, Wu L., Fan J., and Belasco J. G. (2006) Proc Natl Acad Sci USA. 103, 4034-4039 and Eulalio A., Huntzinger E., Nishihara T., Rehwinkel J., Fauser M., and Izaurralde E. (2009) RNA 15, 21-32. Thus, identifying changes in poly(A) tail length can yield insights into mRNA regulation and subsequent physiological impact.

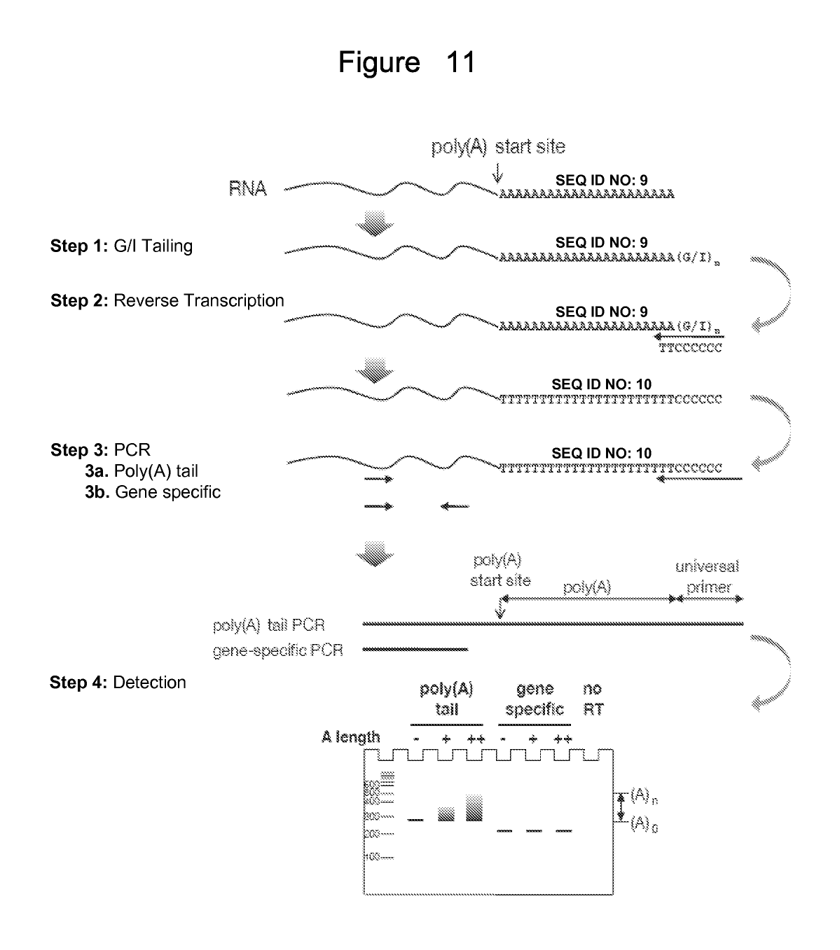

A schematic of the methods used to assay mRNA poly(A) tail length is shown in FIGS. 1 and 11. Briefly, RNA purified from for example unfractionated or fractionated whole cell lysates is treated with a polymerase, for example, purified yeast poly(A) polymerase and GTP:ITP to add a G:I tail to the 3′ end of RNA. Reverse transcription (MMLV reverse transcriptase, USB) was performed with oJC639. Poly(A) tails were detected using oJC640 and a gene specific forward primer for PCR (oJC791 for MFA2 mRNA). In one aspect the GTP:ITP can be 3:1. The sample labeled AO (unadenylated mRNA product) was generated by PCR using oJC789 and oJC790. PCR products were separated on 3% agarose gels followed by staining with SYBRGold (Invitrogen) or ethidium bromide. Stained gels were visualized and imaged using the CHEMIGenius 2 gel dock.

First, total RNA is treated with yeast Poly(A) Polymerase (yPAP) in the presence of high GTP and ITP. Yeast PAP will add a poly(G) tail to the 3′ end of RNA under these conditions. Second, a poly(C) primer is added. In a preferred aspect, this poly(C) primer has two T residues on the 3′ end. Reverse transcription is performed followed by PCR using a message specific primer. Lane (f) Shows a control reaction using the PAP tailing assay. Specifically the MFA2 mRNA was amplified from WT, dcp2Δ (decapping mutant), and ccr4Δ (deadenylase mutant) cells. -PAP: indicates sample in which yPAP was omitted. -RT: indicates sample in which reverse transcriptase was omitted. AO indicates size of completely deadenylated MFA2 mRNA. Size markers are indicated on the outside lanes (in nucleotides). PCR products were resolved on a 3% agarose gel and visualized with SYBRGold (Invitrogen). Lane (g) shows the poly(A) tail status of the MFA2 mRNA across a sucrose gradient in WT cells or dcp2Δ cells (lane (h)). Fractions are indicated above each lane, as are the relative positions of the non-translating region (RNP), 80S monoribosome peak, and polyribosomes peaks. A0 indicates the migration of the fully deadenylated MFA2 mRNA. Size markers are indicated to the left of each gel in nucleotides. The ORF size for MFA2 mRNA is 39 codons.

All reagents for step 1, 2 and 3 reactions, except for RNA sample and PCR forward primer specific for a gene of interest, may be included in a kit. No further changes should be required for component formulations in step 1 poly(A) tail extension reaction and step 2 reverse transcription reaction (USB PN 75780). Step 1 components have passed stability testing for storage at −20° C. for 7 weeks and at 4° C. for 18 days (equivalent to storage at −20° C. for ˜90 days). Step 3 PCR will use HOTSTART-IT® Taq DNA Polymerase. Because the methods are work best with high Mg²⁺ concentrations for optimal PCR condition, we optimized formulations.

In one aspect a method for assaying the length of Poly(A) Tails has been developed (FIG. 1 and Tables 1-3). All reagents for step 1, 2 and 3 reactions, except for RNA sample and forward PCR primer specific for a gene of interest, may be included in a kit. Component formulations in step 1 poly(A) tail extension reaction and step 2 reverse transcription reaction, in a preferred aspect are based on First-Strand cDNA Synthesis Kit for Real-Time PCR (USB PN 75780). Step 3 PCR preferably uses HOTSTART-IT® Taq DNA Polymerase (USB). Because this assay optimally uses high Mg²⁺ concentrations for optimal PCR condition, we disclose herein how to formulate PCR components for the disclosed assays. The assay approach has been validated for measuring poly(A) tail length of RNA from biological samples. The assay components for step 1 of the assay, poly(A) tail extension with G/I are shown in Table 1.

TABLE 1
Step 1 components for 20 μl reaction. Incubate at
37° C. for 60 min and add 1 μl 20X stop solution.

| | Volume for 1 reaction | |
|---|---|---|
| RNA for 1 μg/rxn | X | μl |
| 10X G:I mix | 2 | μl |
| 10X Tail Buffer | 2 | μl |
| 10X yPAP mix | 2 | μl |
| Water | to 20 | μl |

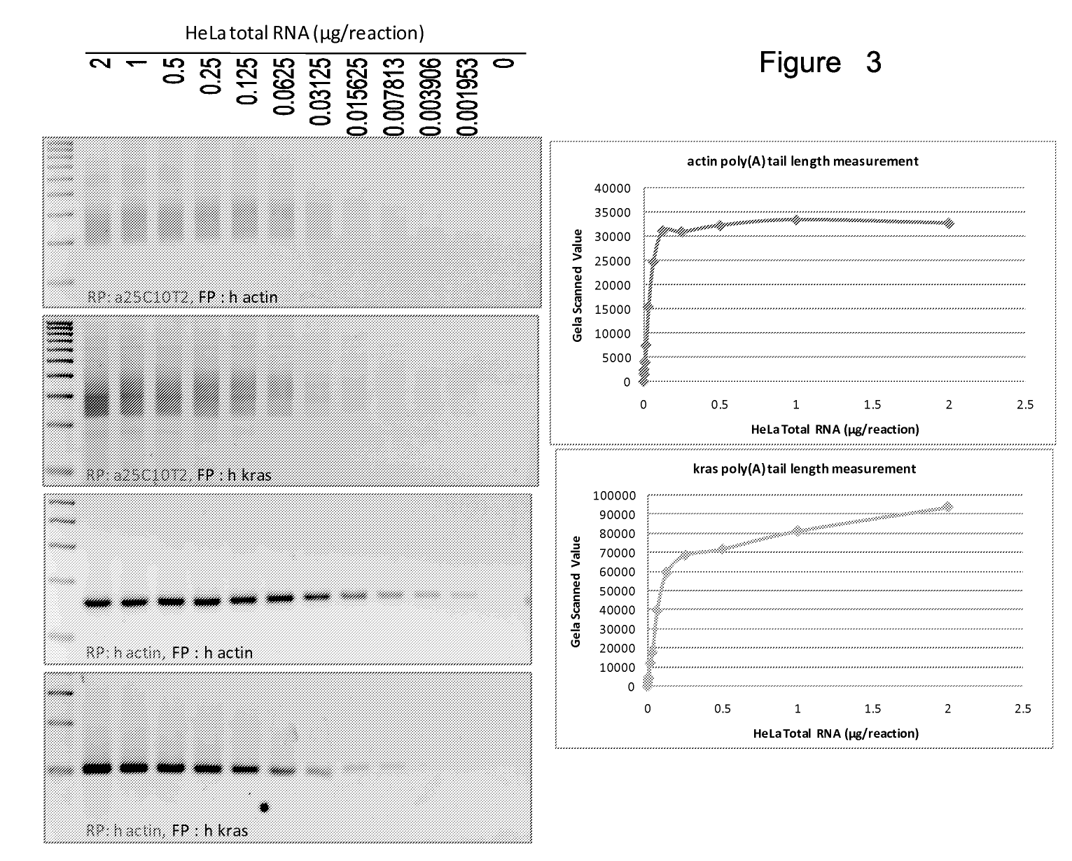

The components in the poly(A) tail extension reaction are sufficient for G/I tailing of all mRNAs present in 1 μg total RNA, the amount of starting material recommended for the assay, and 0.5 μg of in vitro transcribed poly(A) RNA. In FIG. 3 HeLa total RNA was used to determine the assay sensitivity. A two-fold dilution of HeLa total RNA per Poly(A) tail extension reaction shows that this assay can detect poly(A) tail length of actin and k-ras from 8-16 ng total RNA inputs. In preferred aspects about 100 ng or more of total RNA may be used for detection of relatively abundant mRNAs. FIG. 3 shows the sensitivity of the assay. A two-fold serial dilution of HeLa total RNA was used in Poly(A) tail extension reactions. Actin and k-ras poly(A) tail and gene-specific amplifications were determined. The graphs on the right represent scanned values of the poly(A) tail signals on the left.

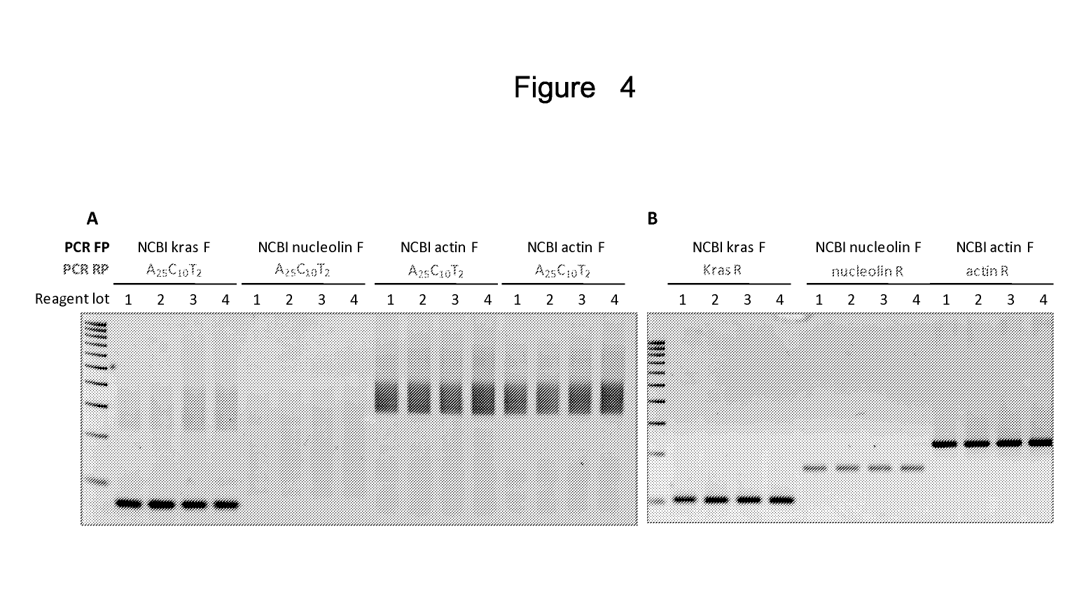

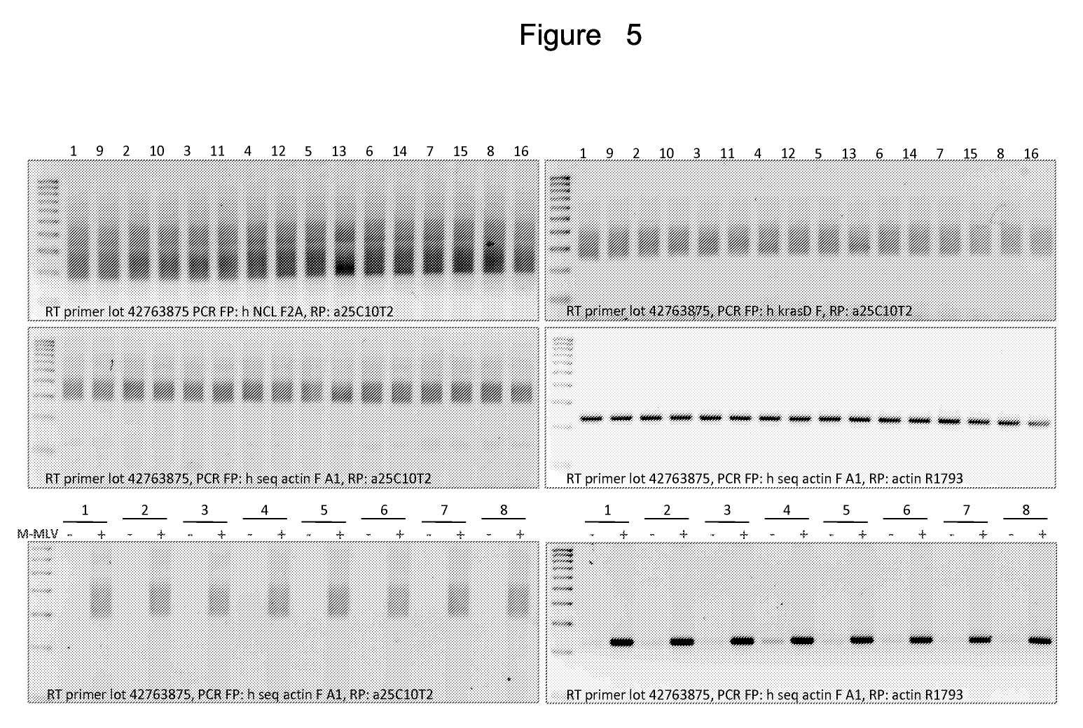

Poly(A) tail extension reaction components were tested for stability by testing after storage at −20° C. for 4 weeks (FIGS. 4A and B) and for 7 weeks and at 4° C. for 18 days (equivalent to storage at −20° C. for ˜90 days) (FIG. 5).

TABLE 2
Step 2: Reverse Transcription 20 μl reaction

| | Volume in μl per reaction |
|---|---|
| 10X RT primer | 2 |
| Tailed RNA | 5 |
| 10X RT Buffer | 2 |
| 10 mM dNTPs | 1 |
| M-MLV RT | 1 |
| RNase inhibitor | 1 |
| RNase free water | To 20 |

Incubate at 44° C. for 60 min then 92° C. for 10 min then store at 4° C. The components may be from First-Strand cDNA Synthesis Kit for Real-Time PCR (USB PN 75780).

TABLE 3
Step 3: PCR Amplification, 25 μl reaction

| | Volume in μl per reaction |
|---|---|
| Diluted RT reaction | 2 |
| 10 μM PCR RP | 1 |
| 10 μM PCR FP | 1 |
| 10 mM dNTPs | 2 |
| 10X PCR buffer | 2..5 |
| 25 mM MgCl₂ | 2.5 |
| HotStart Taq | 1 |
| Water to 25 μl | 13 |

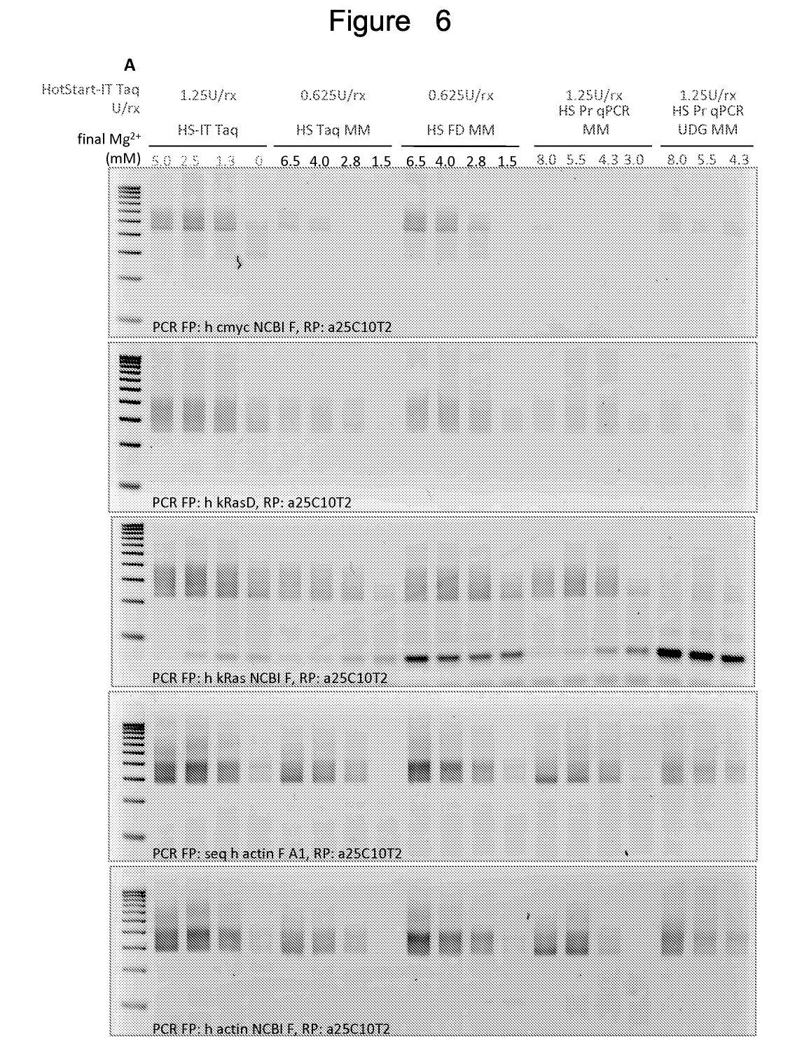

In one aspect, the PCR reaction for this assay uses a HotStart method and Mg²⁺ higher than 1.5 mM. It was also observed that a 2-step PCR cycle (denature and annealing plus extension) gave overall better signal-to-noise ratio than the traditional three-step PCR cycle (denature, annealing, and extension). For added convenience, a HotStart-IT PCR Master Mix was tested in the methods (Table 3 and FIG. 6). The results show that none of the tested PCR Master Mixes performed up to the same levels as the stand alone PN 71195 with Mg²⁺ supplemented in the reaction. It should be noted that reducing HotStart-IT Taq (PN 71195, lot #123643) by 50% resulted in noticeably lower signal.

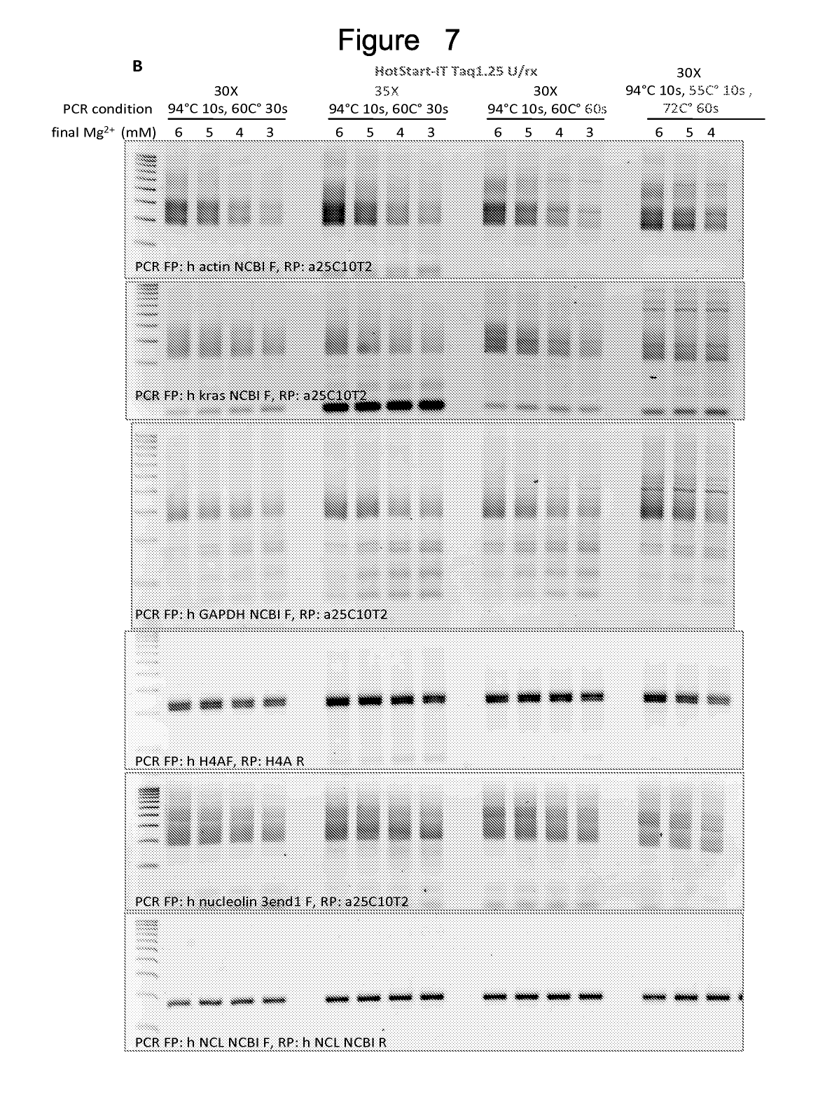

In FIG. 7, the effect of adding additional Mg²⁺ to the standard PCR Reaction Buffer (PN 71165, 15 mM MgCl₂) is shown and a new formulation of 10×PCR Reaction Buffer with 50 mM MgCl₂ is preferred. FIG. 7 also shows the 2-step PCR cycle (30×, 94° C., 10 sec and 60° C., 30 sec) generates less of non-specific amplification compared to the three-step PCR cycle (30×, 94° C., 10 sec, 55° C., 10 sec and 72° C., 60 sec). For the two-step PCR, increasing annealing and extension time from 30 sec to 60 sec did not noticebly improve the signal (FIGS. 6 and 7 set 1 vs. set 3). However, increasing the numbers of cycle to 35 may improve the application of the methods for detection of low expressing targets. Drosophila S2 cell total RNA was also tested in this experiment and showed similar results.

TABLE 4
Mg²⁺, NTPs and Hotstart-IT Taq in USB HotStart-IT Master Mix products.

| | | Final concentration | | | |
|---|---|---|---|---|---|
| | | in 25 μl reaction | | | |
| | | | | HotStart- | |
| | | Mg2+ | NTPs | IT Taq | glycerol |
| PN | Product | (mM) | (mM) | (units) | (%) |
| 71196 | HotStart-IT ® Taq Master Mix (2X) | 1.5 | 0.2 | 0.625 | 5 |
| 71156 | HotStart-IT ® FideliTaq ™ PCR | 1.5 | 0.2 | 0.625 | 5 |
| | Master Mix (2X) | | | | |
| 75766 | HotStart-IT ® Probe qPCR Master | 3 | 0.2 | 1.25 | 5 |
| | Mix (2X) | | | | |
| 75764 | HotStart-IT ™ Probe qPCR Master | 3 | 0.2 | 1.25 | 5 |
| | Mix with UDG (2X) | | | | |
| 71195 | HotStart-IT ® Taq DNA Polymerase | NA | NA | 1.25 | |
| 71165 | 10× PCR Reaction Buffer | 1.5 | 0.2 | NA | |

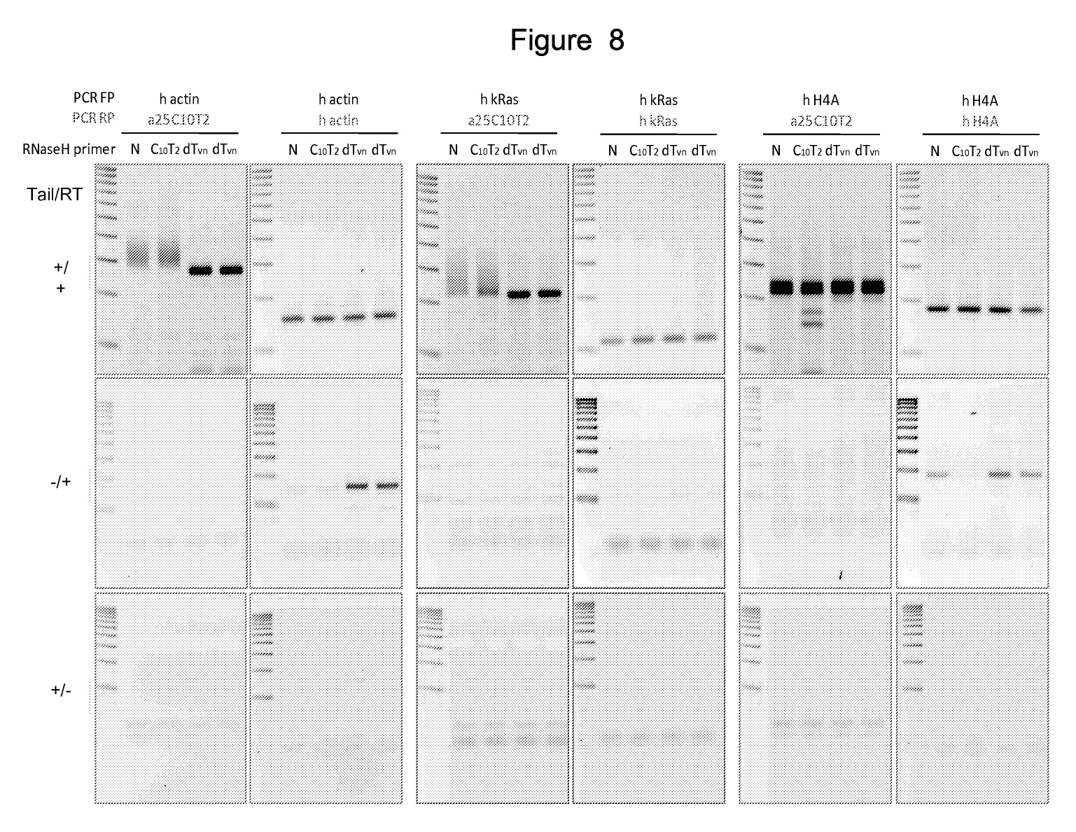

To validate the ability of the methods to accurately measure the length of the poly(A) tail mRNA, the method was tested on total RNA samples of poly(A)-deleted RNA by oligo(dT)/RNase H treatment. Deleting poly(A) by oligo(dT) hybridization and RNase H cleavage is also a negative control for measuring poly(A) tail length by Northern blot assay. In this experiment, N, a no primer control, and C₁₀T₂ primer should result in no hybridization to RNA and therefore the poly(A) tails should remain intact. However, oligo(dT)_{23VN} should hybridize to poly(A) tails, generating sites for RNA cleavage by RNase H and thus removing poly(A) tails from mRNAs. The anchor bases (vn) should direct hybridization to the junction between the end of the 3′ UTR and the start of poly(A) tail, ensuring a complete poly(A) removal. As expected, the results in FIG. 8 show that no poly(A) tail amplification was detected from the sample treated with oligo(dT)_{23VN}, which was shown as a single PCR band compared to the smeared bands of poly(A)-amplified PCR samples (N and C₁₀T₂). Also, as expected, the signals for histone 4A, actin-specific detection and k-ras-specific detection were not affected by the oligo(dT)/RNase H treatment.

FIG. 8 shows Poly(A) tail-length assay of poly(A)-deleted RNA by RNase H/oligo(dT) treatment. Four microgram HeLa total RNA was mixed 400 pmol DNA primers (C10T2 or oligo (dT)_{23VN}), denatured at 75° C. for 5 min and annealed at 4° C. before treatment with 10 units RNaseH at 37° C. for 30 min in a 20 μl reaction volume. The reaction was stopped by adding 2 mM EDTA. Then RNA samples were purified by phenol/chloroform extraction and ethanol precipitation. The amount of RNA in each sample was determined by Nanodrop reader before using 1 μg in poly(A) tail extension reaction.

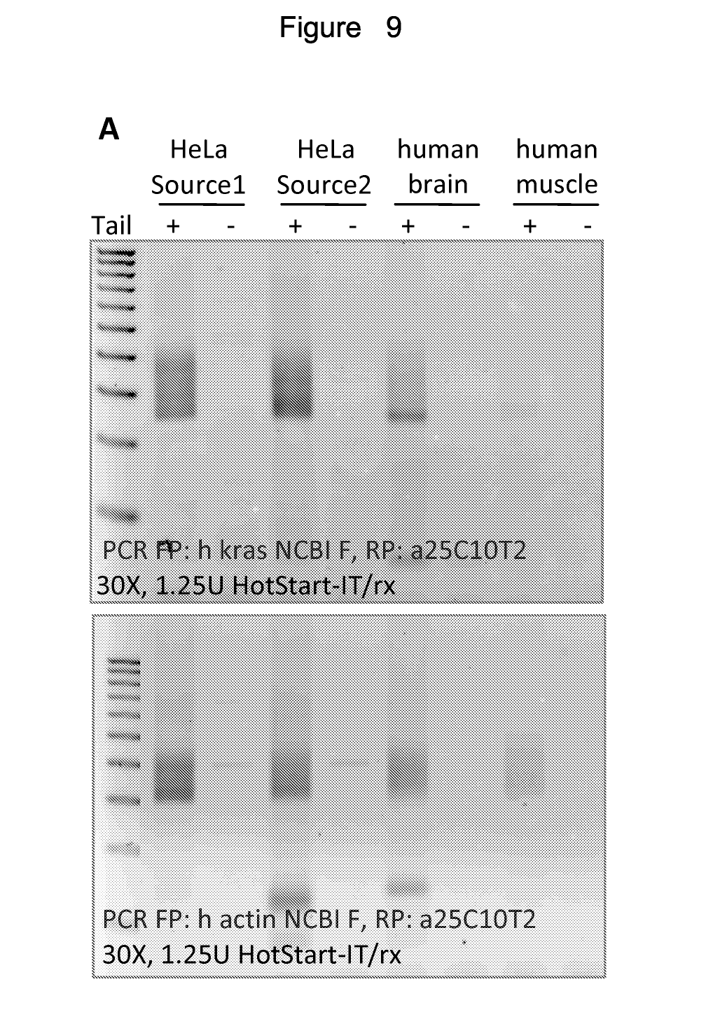

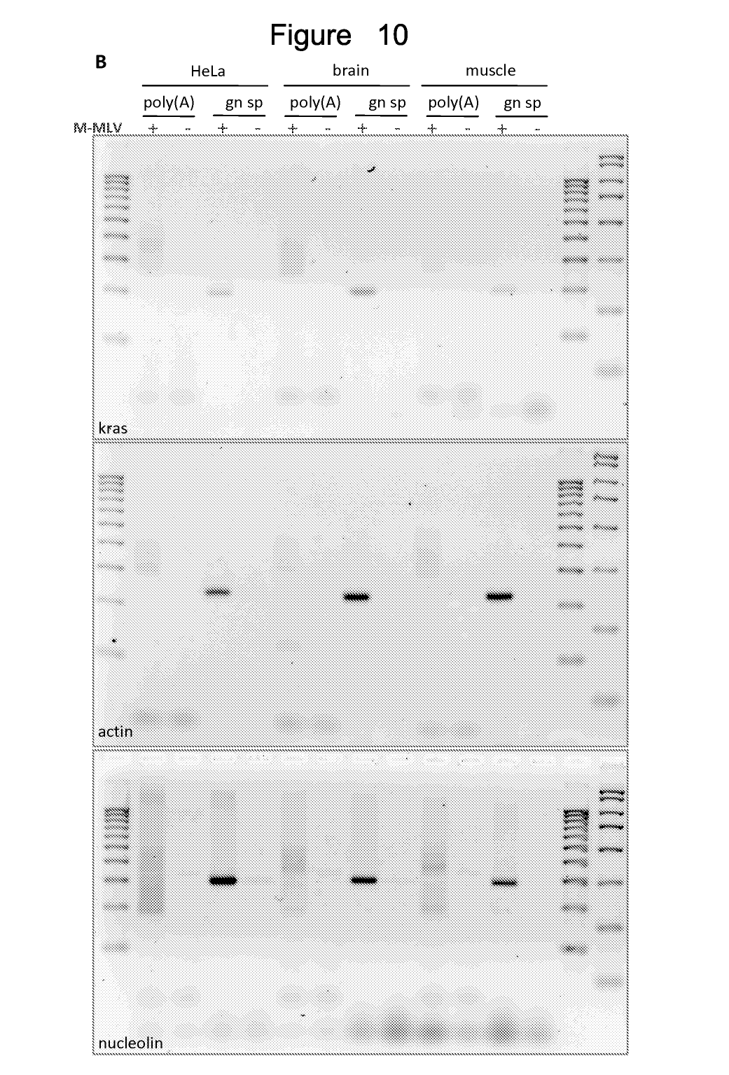

FIGS. 9 and 10, show the use of the Poly(A) Tail-Length Assay for detection of human k-ras, actin and nucleolin poly(A) tails from HeLa, brain and muscle. Similar pattern of actin poly(A) tail was seen in all sample whereas k-ras poly(A) tail was much reduced in muscle. We also see from this experiment that the pattern of nucleolin poly(A) tail is different between HeLa and organ-specific RNAs. FIG. 9 shows the assay used for analysis of human k-ras, actin and nucleolin from HeLa (prepared by TN lab, and purchased from Ambion (AM 7852)), brain (Ambion, AM7962) and muscle (Ambion, AM7982).

The method is able to detect poly(A) tail length and gene-specific detection of actin and k-ras from 0.1 and 1 μg HeLa total RNA (free of genomic DNA) using the standard protocol. The expected PCR products should be visible from half PCR reaction using ethidium bromide-stained 2.5% TAE agarose gel. Products from poly(A) tail length assay sample appear as a mix population of PCR bands representing the length distribution of poly(A) tails in target mRNA. No PCR products should be detected from the negative controls that omit reverse trancriptase at step 2.

The methods and kits disclosed herein use the following four steps to enable poly(A) tail length determination. In Step 1, poly(A) polymerase adds a limited number of guanosine and inosine residues to the 3′-ends of poly(A)-containing RNAs(5,6). See, for example, Martin G., and Keller W. (1998) RNA 4, 226-230 and Kusov Y. Y., Shatirishvili G., Dzagurov G., and Gauss-Müller V. (2001) Nucleic Acids Res. 29, E57-7. In Step 2, the tailed-RNAs are converted to DNA through reverse transcription using the newly added G/I tails as the priming sites. In Step 3, PCR amplification products are generated using two primer sets. A gene-specific forward and reverse primer set designed upstream of the polyadenylation site (e.g. the 3′-UTR) is produced as a control for the gene-of-interest. The second set of primers uses the gene-specific forward primer and the universal reverse primer that is preferably provided with the kit to generate a product that includes the poly(A) tails of the gene-of-interest. Finally, in Step 4, the PCR products are separated on an agarose or polyacrylamide gel. The poly(A) tail-lengths of the gene-of-interest are determined by simple subtraction of the lengths of the poly(A) PCR products from the calculated length of the gene-specific forward primer to the putative polyadenylation start site.

The Poly(A) Tail-Length Assay Kit is designed for G/I tailing up to five samples of total RNA. All necessary components are provided to perform 4 reverse transcription and 80 PCR reactions on each of the five tail-extended samples. Reaction products are then assessed by gel electrophoresis. The protocol includes the following steps: G/I Tailing (˜60 min incubation), reverse transcription (˜70 min incubation), PCR Amplification (30-60 min incubation) and detection.

The following materials may be used in combination with the disclosed methods and may be included with a kit: 100 ng to 2 μg of total RNA, specific PCR forward and reverse primers designed for the gene-of-interest, microcentrifuge, thermal cycler, adjustable precision pipettes, RNase-free filter pipette tips and Nuclease-free tubes, appropriate PCR plates or tubes for instruments, disposable gloves, gel electrophoresis, molecular weight marker (USB PN 76712 or 76710), DNA loading buffer (USB PN 76715 or 76720), 2-2.5% agarose (USB PN 32802) gel and TAE buffer (USB PN 75904 or 74015), 4-6% non-denaturing polyacrylamide (USB PN 75848) and TBE buffer (USB PN 75891) UV transilluminator or fluorescence image scanner.

### Reagent and Sample Handling

Thaw reagents on ice, mix thoroughly before use and immediately return unused materials to −20° C. When preparing working reagents, measure components accurately, mix thoroughly, spin briefly and keep on ice. Assemble reactions on ice or at the indicated temperature throughout the procedure. When working with RNA, wear gloves at all times while handling reagents, materials and equipment to prevent RNase contamination from hands. Clean pipettes and work areas with RNaseAway™ or RNaseZap® to reduce the risk of RNase contamination. Use RNase-free plastic ware and RNase-free buffers and reagents.

### Starting Materials

A typical assay reaction uses 0.1-2 μg of total RNA. The amount of total RNA required per assay depends on the target abundance in the sample. It is important to use RNA that is completely free of contaminating genomic DNA. It is generally unnecessary to treat the RNA with DNase I to remove any genomic DNA contamination. However, certain RNA preparations may yield non-specific amplification products that can be removed by treating the isolated RNA with rDNase I (PN 78311). Samples treated with DNase I should be extracted with phenol-chloroform or purified with a column-based procedure.

### Assay Controls

Prepare an “Assay Positive Control” by using the supplied HeLa Total RNA and human actin PCR Forward Primer. This control will be used to assess assay components and procedures. Prepare a “No RT Negative Control” to assess non-specific amplification by substituting the 10× RT Enzyme Mix with Nuclease-Free Water. Prepare a “Specific Primer Control” to assess specificity of the gene-specific PCR forward primer by substituting the Universal PCR Reverse Primer with the gene-specific PCR reverse primer (not supplied). The following table 5 summarizes the recommended reactions that should be performed.

TABLE 5

| | | | | | | PCR Amplification | | | |
|---|---|---|---|---|---|---|---|---|---|
| | | | | Reverse | | Tail PCR | | Specific | |
| | G/I tailing | | | Transcription | | primers | | primers | |
| | Input | Enzyme | Buffer | Enzyme | Buffer | Forw'd | Rev. | Forw'd | Rev. |
| Positive | HeLa | X | X | X | X | Actin | Universal | N/A | N/A |
| control | RNA | | | | | | | | |

Sample

| | | | | | | | | |
|---|---|---|---|---|---|---|---|---|
| Test | X | X | Water | X | S | Universal | N/A | N/A |
| Test | X | X | X | X | N/A | N/A | S | S |
| Test | X | X | X | X | S | Universal | N/A | N/A |

X indicates use of the component, S indicated gene-specific.

During the Poly(A) Tail-Length Assay, the samples are placed in a thermal cycler three times. Therefore, programming the thermal cycler(s) with the following programs prior to sample processing is preferred.

Exemplary programs are as follows: for G/I Tailing: 37° C. for 60 min, for Reverse Transcription: 44° C. for 60 min; 92° C. for 10 min; and 4° C. hold, for PCR Amplification: a Two-Step PCR or a Three step PCR may be used. For example, 94° C. for 2 min, 30-35 cycles of: 94° C. for 10 sec, 60° C. for 30-60 sec, 72° C. for 5 min, 4° C. hold OR 94° C. for 2 min, 30-35 cycles of: 94° C. for 10 sec, 58° C. for 30 sec, 72° C. for 30 sec, 72° C. for 5 min, 4° C. hold. Certain targets may exhibit sub-optimal amplification with the Two-Step PCR protocol. The Three-Step PCR protocol may be used in cases where weak PCR amplification is observed.

### Protocol Step 1: G/I Tailing

The following protocol may be used to add poly(G/I) tails to a total RNA sample. For the positive control, substitute the provided HeLa total RNA for an experimental sample. This standard protocol applies to a single 20 μl G/I Tailing reaction. Thaw frozen reagents on ice and mix thoroughly by vortexing. Enzyme mixes should be gently flicked to mix. Centrifuge briefly. Add the following reagents in Table 6 to a nuclease-free tube. Mix gently by pipetting up and down and then centrifuge the tube briefly to collect the contents. Keep samples on ice.

TABLE 6
G/I tailing mix

| Reagent | Per reaction | |
|---|---|---|
| Total RNA sample 1 μg (0.1-2 μg) | Up to 14 | μl |
| 5X tail buffer mix | 4 | μl |
| 10X tail enzyme mix | 2 | μl |
| Water, nuclease free | To 20 | μl |

Incubate at 37° C. for 60 min. Add 2 μl 10× Tail Stop Solution and mix well. Proceed to Step 2: Reverse Transcription. Alternatively, tailed RNA samples can be stored at −20° C. until ready to proceed to Step 2.

### Step 2: Reverse Transcription

Use the following protocol to reverse transcribe the poly(G/I) tailed RNA. This standard protocol applies to a single 20 μl reverse transcription reaction. Master mixes for multiple reactions can be made by increasing the volumes of reaction components proportionally. Thaw frozen reagents on ice and mix thoroughly by vortexing. Enzyme mixes should be gently flicked to mix. Centrifuge briefly. Add the following reagents in Table 7 to a nuclease-free tube. Mix gently and briefly spin down the tube contents. Keep on ice.

TABLE 7
RT Mix

| Reagent | RT plus | RT minus control |
|---|---|---|
| G/I tailed RNA sample | 5 μl | 5 μl |
| 5X RT Buffer Mix | 4 μl | 4 μl |
| 10X RT enzyme mix | 2 μl | — |
| Water, nuclease free | 9 μl | 11 μl |

Incubate at 44° C. for 60 min; 92° C. for 10 min; and at 4° C. hold. Proceed to Step 3: PCR Amplification. Alternatively, cDNA samples can be stored at −20° C. until ready to proceed to Step 3.

### Step 3: PCR Amplification

Use the following protocol to PCR amplify the poly(G/I) tailed cDNA. This standard protocol applies to a single 25 μl PCR reaction. Master mixes for multiple reactions can be made by increasing the volumes of reaction components proportionally. Dilute each RT sample by adding 20 μl Nuclease-Free Water (40 μl final volume). Thaw frozen reagents on ice and mix thoroughly by vortexing. Mix HOTSTART-IT® Taq DNA Polymerase by gently flicking Centrifuge briefly. Add the following reagents in Table 8 to a nuclease-free tube. Mix gently and briefly spin down the tube contents. Keep on ice.

TABLE 8

| | RT + Tail | | RT − Tail | | RT + | | RT − | |
|---|---|---|---|---|---|---|---|---|
| | PCR | | PCR | | specific | | Specific | |
| Diluted RT sample | Up to 5 | μl | Up to 5 | μl | Up to 5 | μl | Up to 5 | μl |
| 5X PCR Buffer Mix | 5 | μl | 5 | μl | 5 | μl | 5 | μl |
| 10 μM Gene-specific PCR for'd | 1 | μl | 1 | μl | 1 | μl | 1 | μl |
| primer | | | | | | | | |
| 10 μM universal PCR reverse primer | 1 | μl | 1 | μl | — | | — | |
| 10 μM Gene-Specific rev. primer | — | | — | | 1 | μl | 1 | μl |
| 25 mM MgCl₂ | Optional | | Optional | | Optional | | Optional | |
| 1.25 units/μl HotStart-IT Taq DNA | 1 | μl | 1 | μl | 1 | μl | 1 | μl |
| polymerase | | | | | | | | |
| Water nuclease free | To 25 | μl | To 25 | μl | To 25 | μl | To 25 | μl |

Additional MgCl₂ may be required to increase amplification efficiency of certain targets and may be provided in a kit. 4. Proceed to Step 4: Detection. Alternatively, PCR products can be stored at −20° C. until ready to proceed to Step 4.

### Step 4: Detection

The size of PCR products can be assessed by running an aliquot of the reaction on an agarose or polyacrylamide gel. To start, one half of each PCR reaction (12.5 μl) may be loaded per lane on a 2.5% agarose TAE gel. For increased resolution, load one half of each PCR reaction (12.5 μl) per lane on a 5% non-denaturing polyacrylamide TBE gel. Stain gels with ethidium bromide or SYBR® Gold and visualize with a standard ultraviolet transilluminator or fluorescence image scanner. See the Supplementary Information Section for guidelines on gel electrophoresis and data analysis.

### Data Analysis

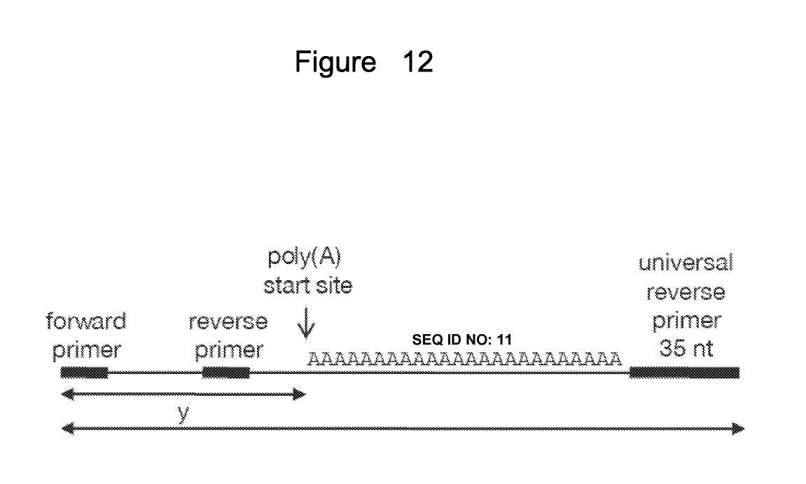

The methods and kits disclosed are useful for determining the length distribution of mRNA poly(A) tails. PCR products of mRNAs with short tails will yield discrete bands, whereas mRNAs with long tails will yield a smear on the gel (FIG. 11). PCR amplification with the gene-specific forward primer and Universal reverse primer amplifies the sequence upstream of the polyadenylation start site site (e.g. the 3′-UTR) to the end of the poly(A) tails. The poly(A) tail-lengths of the gene-of interest are the sizes of poly(A) PCR-amplified products minus the calculated length of the gene-specific forward primer to the putative polyadenylation start site (FIG. 12). PolyA-tail length is (z-y-35) where z can vary based on gel results. The 35 in the equation is the length of the universal primer and that can be varied as well and the equation would similarly change.

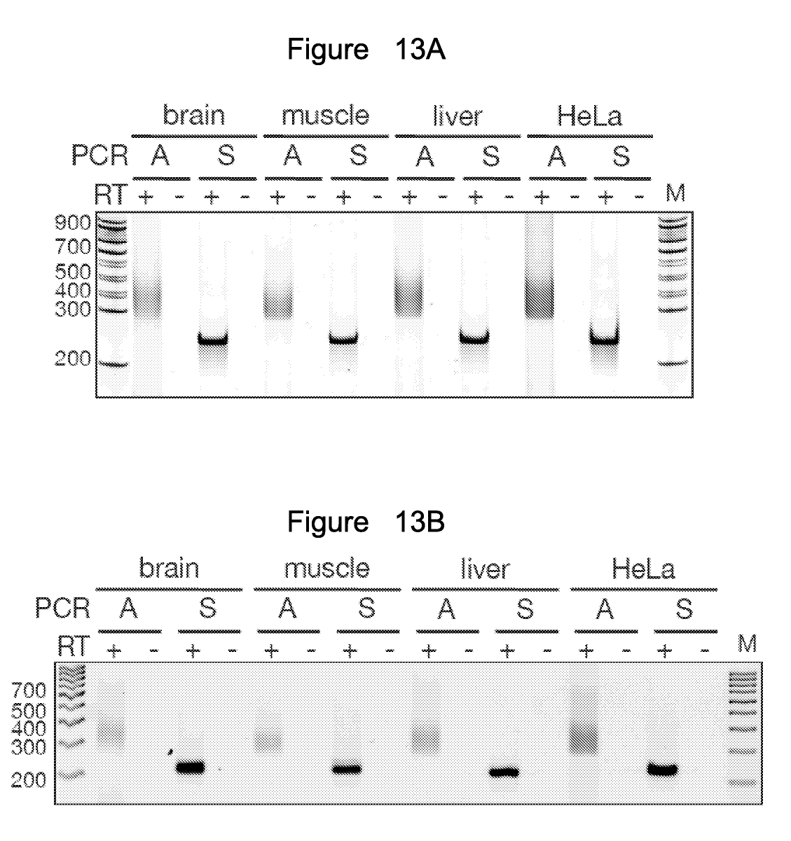

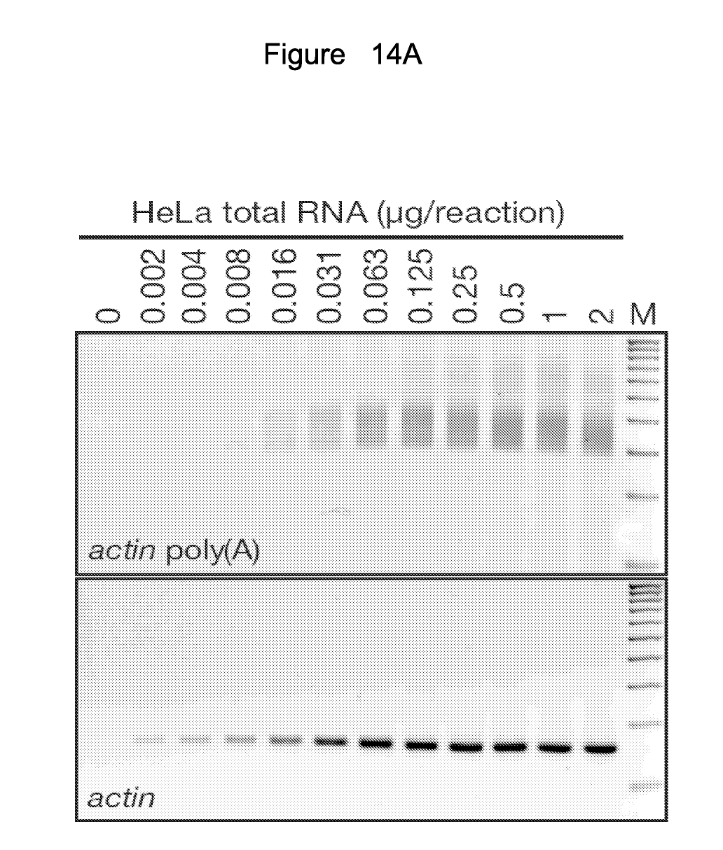

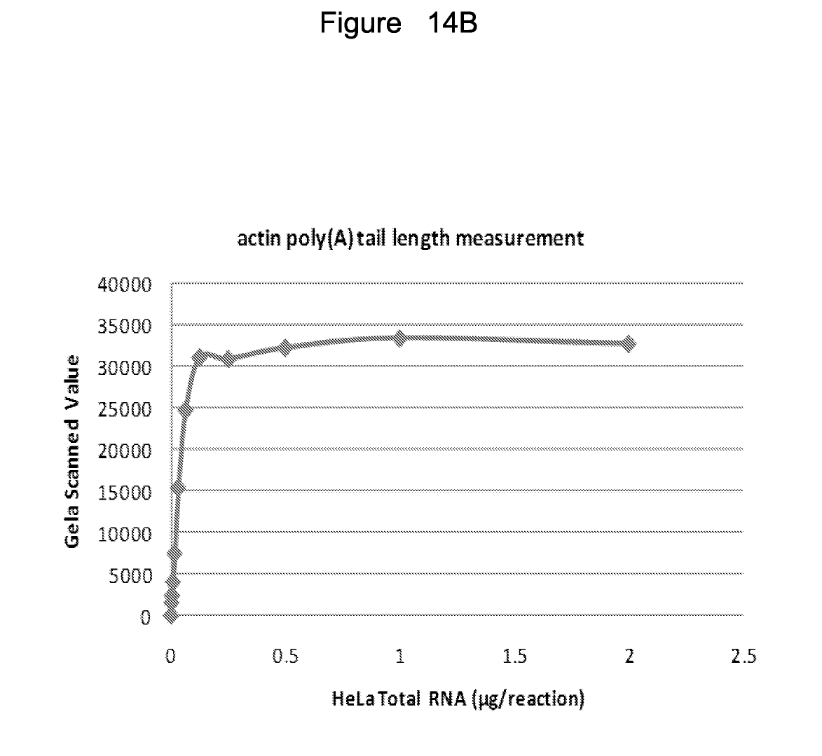

PCR with the gene-specific forward and reverse primers should amplify only the upstream sequence of the expected size to validate the specificity of the gene-specific forward primer. The “No RT Negative Control” reaction should have no signal. Examples of results are shown in FIGS. 13 and 14.

FIG. 13 shows a comparison of human actin poly(A) tail-lengths in brain, muscle, liver and HeLa cell. One microgram total RNA and 4 μl of diluted RT samples were used in G/I Tailing and PCR reactions, respectively. The recommended two-step PCR program was used. One half of each PCR reaction (12.5 μl) was analyzed on 6% non-denaturing polyacrylamide-TBE gel stained with SYBR® Gold (A), and 2.5% agarose-TAE gel stained with ethidium bromide (B). RT (+); No RT Negative Control (−); poly(A) tail PCR (A); genespecific PCR (S); and 100 bp DNA Ladder (USB PN 76712) (M).

FIG. 14 shows detection sensitivity of the Poly(A) Tail-Length Assay. Actin poly(A) tail-length was determined from a two-fold serial dilution HeLa total RNA. Samples were processed as described in FIG. 13B (A). The top image was quantified by densitometry (B).

### PCR Primer Design

In preferred aspects a niversal reverse primer may be used: The Universal PCR Reverse Primer may be supplied as a component of a kit and may be used as the reverse primer in poly(A) tail-length detection PCR reactions. It may be supplied, for example, at 10 μM and used at a final concentration of 400 nM.

Gene-specific forward and reverse primers: These are the primers that are user defined for the gene-of-interest. They should be diluted to 10 μM in TE Buffer (PN 75893) and used at a final concentration of 400 nM. The forward primer is used with the universal reverse primer to generate the poly(A) tail-length PCR products and the gene-specific forward and reverse primers are used together to verify the specificity of the forward primer and the presence of the target within the RNA sample.

The gene-specific PCR primers should be located within 50-300 nucleotides upstream of the poly(A) start site to allow proper resolution of PCR products by gel electrophoresis. If possible, the gene-specific reverse primer should be located immediately upstream of the poly(A) start site for straightforward calculation of the poly(A) tail-lengths. In preferred aspect it a computer programs designed to select appropriate primers in a given sequence is used. Several public primer databases are available on the internet. Some examples of databases are available from NCBI, frodo at whitehead/MIT and from IDT (primerquest).

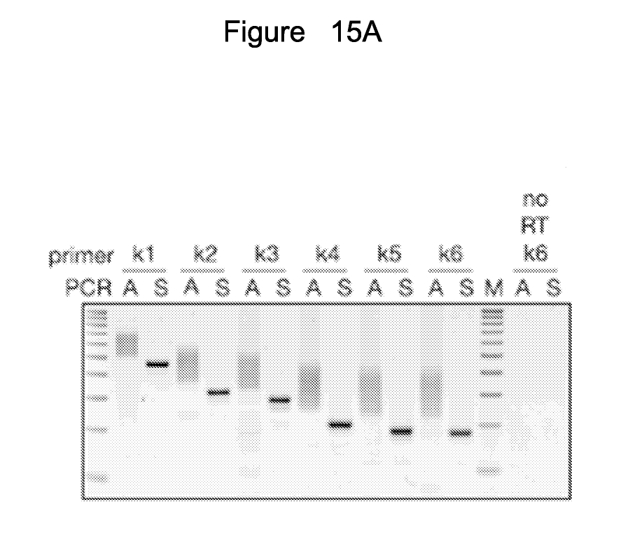

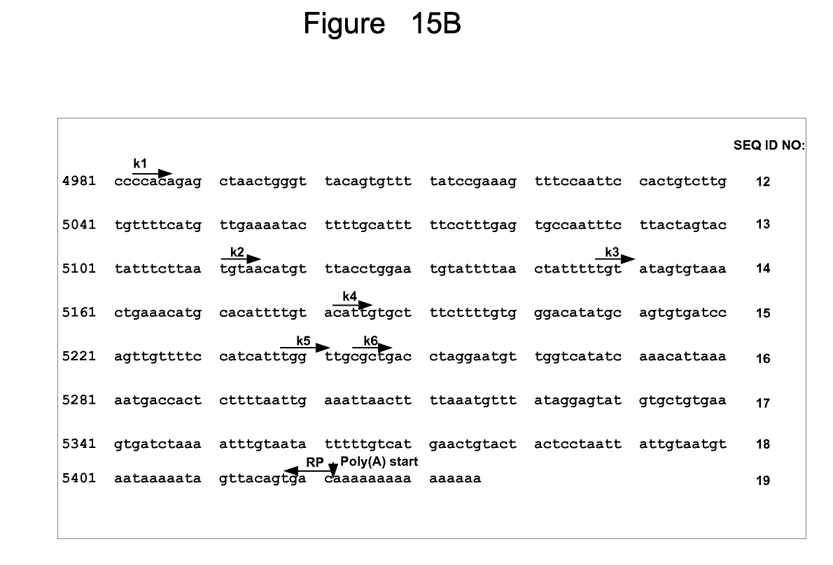

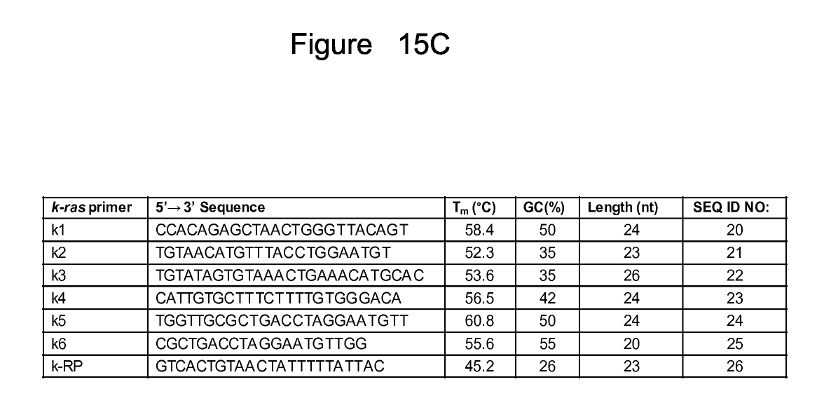

In general, best results are obtained for the disclosed methods when the following guidelines are used: •Primers should range in length from 19 to 30 nucleotides, •G+C content in the range of 30 to 50%, •Tm values ranging from 55-60° C., •Analyze for cross-reactivity in the organism's database. Due to the AT-rich content in 3′ UTR sequences, it may be difficult in some cases to design a primer that fits these specifications. Primers with Tm below 55° C. and have been tested and found to work in the disclosed assay methods best provided the gene-specific forward primer has been validated for specific priming and amplification of the gene-of-interest. In general, two specific forward primers and one specific reverse primer should be designed per gene-of-interest for best possible results. An example of using different specific forward primer designs for poly(A) tail-length determination is shown in FIG. 15.

The tail length in a preferred aspect is analyzed using gel electrophoresis. The gel may be, for example, agarose or polyacrylamide or combinations thereof. Any method for size separation may be used. For an agarose gel the following protocol may be used. Choose a horizontal gel electrophoresis apparatus with a capacity of ≧15 μl per well. Prepare 2.5% agarose TAE gel by mixing 2.5 gm agarose (PN 32802) per. 100 ml 1×TAE Buffer (e.g. PN 75904 or 74015, diluted to 1× with distilled water). Heat to boil the agarose until completely dissolved. Cool to ˜65° C., then add ethidium bromide to 1 μg/ml (or 1 drop of ethidium bromide, PN 75816, per 100 ml). Pour the gel solution into the gel tray with comb to form wells and let set completely. Prepare sample by adding loading buffer to 1× (e.g. 4 μl of 6× Loading Buffer, PN 76715 or PN 76720). Mix and quick spin to collect tube contents at the bottom of the tubes. Load 14 μl of the dye-PCR mix sample per lane. For the first and the last lane, load DNA marker (e.g. 3 μl of 100 bp DNA Ladder, PN 76712). Run in 1×TAE Buffer (e.g. PN 75904, diluted to 1× with distilled water) at 150 volts for 40-60 min. Visualize and document with a standard ultraviolet transilluminator or fluorescence image scanner.

For a polyacrylamide gel the following protocol may be used. Choose a vertical gel electrophoresis apparatus with a capacity of ≧15 μl per well. Follow the manufacturer's instructions for the details of assembling gel apparatus. One 10 cm×15 cm×1 mm gel requires 15 ml of gel solution. Prepare 5% polyacrylamide TBE gel by mixing 3 ml 5×TBE (PN 75891), 1.9 ml 40% acrymlamide solution (19:1 acrylamide:bis-acrylamide, PN 75848), 10.1 ml water (to 15 ml total) and immediately before pouring the Pour the gel solution into the gel cassette and place comb to form wells and let polymerize completely at room temperature for at least 30 min. Prepare sample by adding loading buffer to 1× (e.g. 4 μl of 6× Loading Buffer, PN 76715 PN 76720). Mix and quick spin to collect tube contents at the bottom of the tubes. Load 14 μl of the dye-PCR mix sample per lane. For the first and the last lane, load DNA marker (e.g. 3 μl of DNA Ladder, 100 bp, USB PN 76712). Run in 1×TBE Buffer (e.g. PN 75891, diluted to 1× with distilled water) at. ˜7 watt, constant power or ˜25 mAmp, constant current for 30-60 min. Stain with SYBR® Gold Nucleic Acid Gel Stain (Life Technologies) according to the manufacturer's instructions. Visualize and document with a standard ultraviolet transilluminator or fluorescence image scanner.

FIG. 15 shows different gene-specific forward primer designs for poly(A) tail-length determination of k-ras from HeLa total RNA. Primer location on k-ras transcript (FIG. 15B) and primer information are shown (FIG. 15C). Samples were processed as described in FIG. 13B (A). No RT Negative Control (RT−); poly(A) tail PCR (A); gene-specific PCR (S); and 100 bp DNA Ladder (USB PN 76712) (M).

### CONCLUSION

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

We claim:

1. A method for estimating the length of the polyA tail of a target mRNA comprising:
adding a guanosine-inosine tail to the 3′ end of the target mRNA;
hybridizing a first primer to the guanosine-inosine tail and extending said first primer to obtain a primer extension product comprising a complement of the polyA tail of the target mRNA;
amplifying the primer extension product by PCR using a universal primer and a target specific primer to obtain a PCR product;
estimating the length of the PCR product; and,
estimating the length of the polyA tail of the target mRNA from the estimated length of the PCR product.

2. The method of claim 1 wherein the first primer is SEQ ID NO. 1.

3. The method of claim 1 wherein the universal primer consists of SEQ ID NO. 1 and a priming sequence that is 5′ of SEQ ID NO. 1 so that the 3′ end of SEQ ID NO. 1 is the 3′ end of the universal primer.

4. The method of claim 3 wherein the priming sequence is between 15 and 30 bases.

5. The method of claim 3 wherein the priming sequence is between 18 and 26 bases.

6. The method of claim 1 wherein the guanosine-inosine tail is added by a polyA polymerase.

7. The method of claim 6 wherein the polyA polymerase is a yeast polyA polymerase.

8. A kit comprising a reverse transcription primer that comprises between 2 and 5 contiguous T bases at the 3′ end and between 8 and 15 contiguous C bases at the 3′ end.

9. The kit or claim 8 further comprising one or more components selected from a mixture of guanosine and inosine, a poly(A) polymerase, reverse transcriptase, thermostable polymerase, buffer, RNase inhibitor, nuclease free water, one or more gene specific primers, and a universal primer.

10. The kit of claim 8 comprising a plurality of target specific primers, targeting a plurality of different target mRNAs.

11. The kit if claim 9 comprising a plurality of target specific primers, targeting a plurality of different target mRNAs.

12. The kit of claim 9 comprising 3 to 50 different target specific primers each targeting a different target mRNA.

13. The kit of claim 9 wherein the reverse transcription primer is SEQ ID NO. 1 and the universal primer is SEQ ID NO. 2.

14. The method of claim 1 wherein the polyA tail length of 2 to 50 different mRNAs are estimated.

15. The method of claim 1 wherein the universal primer is SEQ ID NO. 2.
